(19) United States
(12) Patent Application Publication
(10) Pub. No.: US 2011/0257686 A1
(43) Pub. Date: Oct. 20, 2011

(54) FLEXIBLE BONE FASTENER AND METHODS OF USE

(75) Inventors: Newton H. Metcalf, JR., Memphis, TN (US); Gregory C. Marik, Collierville, TN (US)

(73) Assignee: WARSAW ORTHOPEDIC, INC., Warsaw, IN (US)

(21) Appl. No.: 12/762,032

(22) Filed: Apr. 16, 2010

(51) Int. Cl. A61B 17/70; A61B 17/86

(52) U.S. Cl. 606/264; 606/310

(57) ABSTRACT

A bone fastener includes an elongated first section having a distal end. A flexible member has an outer surface and defines an inner cavity configured for disposal of the distal end of the first section. An elongated second section defines a longitudinal axis and includes a wall that defines an interior cavity configured for disposal of the flexible member such that the distal end of the first section is disposed within the interior cavity of the second section. The outer surface of the flexible member engages the wall in a configuration such that the first section is pivotable to a plurality of axial orientations relative to the longitudinal axis of the second section.

### TECHNICAL FIELD

The present disclosure generally relates to medical devices for the treatment of bone disorders, and more particularly to a bone fastener that includes a flexible component configured to minimize stress in a vertebral rod system and facilitate motion to prevent fastener failure.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders include discectomy, laminectomy, fusion and implantable prosthetics. As part of these surgical treatments, spinal constructs such as vertebral rods are often used to provide stability to a treated region. Rods redirect stresses away from a damaged or defective region while healing takes place to restore proper alignment and generally support the vertebral members. During surgical treatment, one or more rods may be attached via fasteners to the exterior of two or more vertebral members. This disclosure describes an improvement over these prior art technologies.

### SUMMARY OF THE INVENTION

Accordingly, a bone fastener is disclosed that includes a flexible component, which is configured to minimize stress in a vertebral rod system and facilitate motion to prevent fastener failure.

In one particular embodiment, in accordance with the principles of the present disclosure, a bone fastener is provided. The bone fastener includes an elongated first section having a distal end. A flexible member has an outer surface and defines an inner cavity configured for disposal of the distal end of the first section. An elongated second section defines a longitudinal axis and includes a wall that defines an interior cavity configured for disposal of the flexible member such that the distal end of the first section is disposed within the interior cavity of the second section. The outer surface of the flexible member engages the wall in a configuration such that the first section is pivotable to a plurality of axial orientations relative to the longitudinal axis of the second section.

In one embodiment, the bone fastener includes a shank having an outer surface, a proximal end and a distal end. A flexible member has an outer surface and an inner surface defining an inner cavity having a closed distal end. The inner cavity is configured for disposal of the distal end of the shank. The inner surface engages the outer surface of the shank adjacent the distal end. A penetrating member defines a longitudinal axis and includes a circumferential wall that defines an interior cavity configured for disposal of the flexible member such that the distal end of the shank is disposed within the interior cavity of the penetrating member. The outer surface of the flexible member engages the wall such that the flexible member is configured to provide damping.

In one embodiment, a vertebral construct is provided. The vertebral construct includes at least two bone fasteners, similar to those described herein, and at least one vertebral rod having a first end and a second end. The first end is supported with a cavity of a proximal end of a first bone fastener and the second end is supported with a cavity of a proximal end of a second bone fastener.

### BRIEF DESCRIPTION OF THE DRAWINGS

The present disclosure will become more readily apparent from the specific description accompanied by the following drawings, in which:

FIG. 1 is a side view, in part cross section, of one particular embodiment of a bone fastener in accordance with the principles of the present disclosure;

FIG. 2 is an enlarged, cut-away side view in part cross-section of the bone fastener shown in FIG. 1;

FIG. 3 is a side view of one particular embodiment of a vertebral construct, including the bone fastener shown in FIG. 1, attached to vertebrae in accordance with the principles of the present disclosure; and

FIG. 4 is a plan view of the vertebral construct shown in FIG. 3 attached to vertebrae.

Like reference numerals indicate similar parts throughout the figures.

### DETAILED DESCRIPTION OF THE INVENTION

The exemplary embodiments of the bone fastener and methods of use disclosed are discussed in terms of medical devices for the treatment of bone disorders and more particularly, in terms of a bone fastener that includes a flexible component configured to minimize stress to a bone construct and adjacent tissues, including bone, for applications such as, for example, a vertebral rod system. The flexible component of the bone fastener facilitates motion to prevent fastener failure, which may include fastener fracture and/or loosening. It is envisioned that employment of the bone fastener with a vertebral rod system provides stability and maintains structural integrity while reducing stress on spinal elements. The flexible bone fastener may also be used with other constructs such as plates. It is contemplated that a bone construct may include the bone fastener only, in for example, fracture repair such as femur and arthroscopy applications.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures, of such disorders, such as, for example, discectomy, laminectomy, fusion, bone graft, implantable prosthetics and/or dynamic stabilization applications. It is contemplated that the present disclosure may be employed with other osteal and bone related applications, including those associated with diagnostics and therapeutics. It is further contemplated that the disclosed bone fastener may be employed in a surgical treatment with a patient in a prone or supine position, employing a posterior, lateral or anterior approach. The present disclosure may be employed with procedures for treating the lumbar, cervical, thoracic and pelvic regions of a spinal column.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

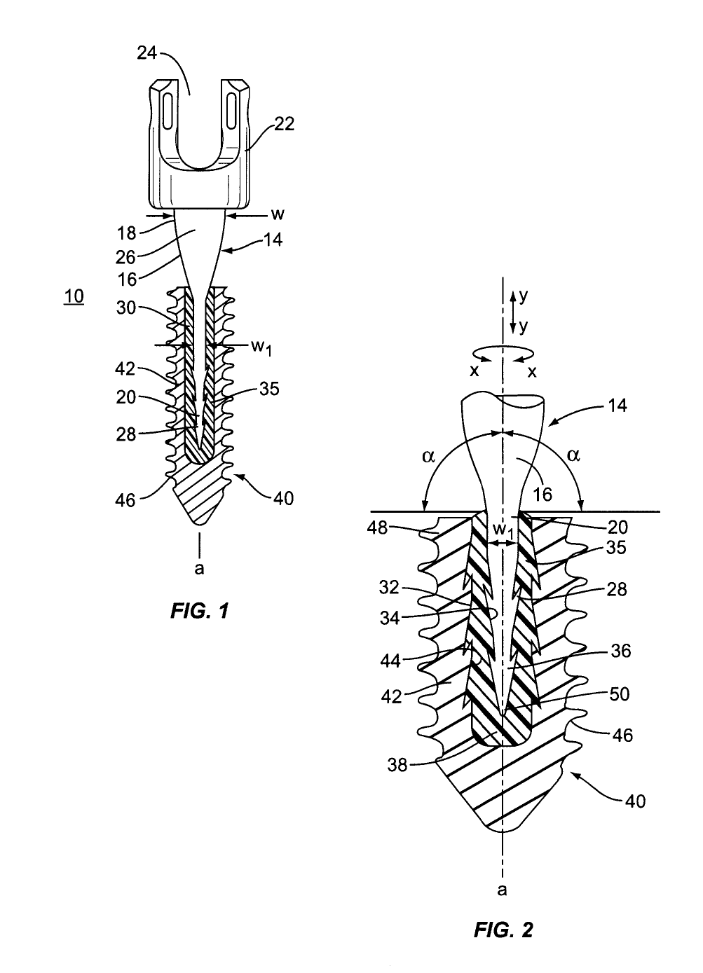

The following discussion includes a description of a bone fastener, related components and exemplary methods of employing the bone fastener in accordance with the principles of the present disclosure. Alternate embodiments are also disclosed. Reference will now be made in detail to the exemplary embodiments of the present disclosure, which are illustrated in the accompanying figures. Turning now to FIG. 1, there is illustrated components of a bone fastener 10 in accordance with the principles of the present disclosure.

The components of bone fastener 10 and bone constructs, such as, for example, a vertebral rod system employed therewith, are fabricated from materials suitable for medical applications, including metals, polymers, ceramics, biocompatible materials and/or their composites, depending on the particular application and/or preference of a medical practitioner. For example, bone fastener 10 and/or a vertebral rod 12, discussed below, of the vertebral rod system can be fabricated from materials such as commercially pure titanium, titanium alloys, Grade 5 titanium, super-elastic titanium alloys, cobalt-chrome alloys, stainless steel alloys, superelastic metallic alloys (e.g. Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof such as calcium phosphate (e.g. SKELITE™ manufactured by Biologix Inc.), rigid polymers including polyphenylene, polyamide, polyimide, polyetherimide, polyethylene, polyurethanes of any durometer, epoxy and silicone. Different components of the vertebral rod system may have alternative material composites to achieve various desired characteristics such as strength, rigidity, elasticity, compliance, biomechanical performance, durability and radiolucency or imaging preference. The components of the vertebral rod system may also be fabricated from a heterogeneous material such as a combination of two or more of the above-described materials.

It is envisioned that the components of the vertebral rod system can be manufactured via various methods. For example, bone fastener 10 can be manufactured and assembled via injection-molding, insert-molding, overmolding, compression molding, transfer molding, co-extrusion, pultrusion, dip-coating, spray-coating, powder-coating, porous-coating, machining, milling from a solid stock material, and their combinations. One skilled in the art, however, will realize that such materials and fabrication methods suitable for assembly and manufacture, in accordance with the present disclosure, would be appropriate.

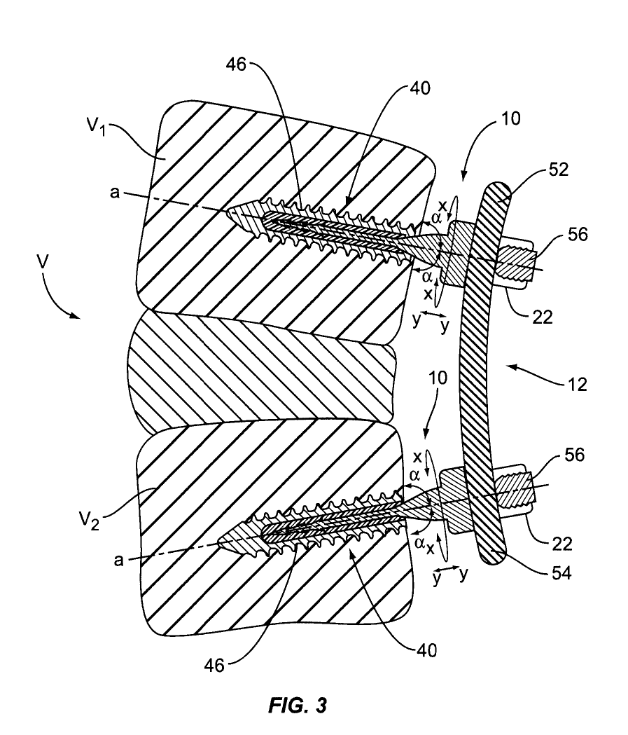

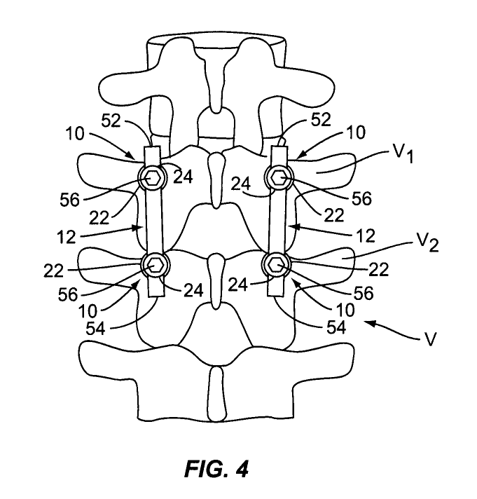

Bone fastener 10 is employed with a vertebral rod system, which is configured for attachment to bone, such as, for example, vertebrae V (as shown, for example, in FIGS. 3-4) during surgical treatment of a spinal disorder, examples of which are discussed herein. Bone fastener 10 includes an elongated first section, such as, for example, a shank 14 having an outer surface 16, a proximal end 18 and a distal end 20. Shank 14 has a cylindrical shaft configuration. Proximal end 18 includes a head 22 configured to support a bone construct, such as, for example, vertebral rod 12. Head 22 includes a bore or through opening 24 configured to support rod 12.

Shank 14 has a body 26 that extends from proximal end 18 to distal end 20. Body 26 tapers from a first thickness having a diameter w to a second, reduced thickness having a diameter w₁. It is contemplated that shank 14 or only portions thereof can be variously dimensioned, for example, with regard to length, width, diameter and thickness. It is further contemplated that the cross-sectional geometry or only portions thereof may have, various configurations, for example, round, oval, rectangular, irregular, consistent, variable, uniform and non-uniform.

Distal end 20 includes locking elements, such as, for example, barbs 28. Barbs 28 fix distal end 20 with a flexible member 30 to facilitate flexible movement of shank 14, as will be described. It is envisioned that shank 14 may include alternate locking or fastening elements to fix distal end 20 with flexible member 30 such as integral connection, threaded engagement, clips, friction fit, interference fit, pins and/or adhesive.

Flexible member 30 has a cylindrical, tubular configuration and defines an outer surface 32 and an inner surface 34. Inner surface 34 defines an inner cavity 36 having a closed distal end 38. It is envisioned that distal end 38 may be open, slotted, perforated and/or planar.

Inner cavity 36 is configured for disposal of distal end 20. Inner surface 34 has a continuous, non-interrupted configuration and is disposed in close fitting engagement with outer surface 16. It is contemplated that inner surface 34 may be non-continuous and interrupted, such as, for example, slotted, perforated, dimpled and/or undulating.

Inner surface 34 engages outer surface 16 such that barbs 28 fix shank 14 with flexible member 30. Inner surface 34 and outer surface 32 define a wall 35 having a thickness and that is circumferentially disposed about the entire outer surface 16 of distal end 20. It is contemplated that flexible member 30 can be variously dimensioned, for example, with regard to the length or thickness of wall 35, and cross sectional geometry such as those discussed above. For example, the cross-sectional geometries of outer surface 32 and/or inner surface 34 can be round, oval, rectangular, irregular, consistent, variable, uniform and non-uniform, and surfaces 32, 34 may have the same or different cross section geometry.

Member 30 is flexible relative to shank 14 and second section 40, discussed below, to provide relative movement and dampening resistance to movement of shank 14 and/or section 40. It is envisioned that flexible member 30 may provide increasing, variable, constant and/or decreasing resistance.

Flexible member 30 may also have one or a plurality of elements connecting sections 14, 40 such as spaced apart portions, staggered patterns and mesh. Flexible member 30 has a different material property such as strength, modulus and flexibility relative to sections 14, 40. It is envisioned that particular parameters of flexible member 30 may be selected to modulate the flexibility or stiffness of the vertebral rod system including the material modulus that may correlate to hardness and modification of porosity, which may include modification of void volume. For example, the fabrication material selected for member 30 provides a range of flexibility according to the requirements of a particular application. Member 30 can be fabricated from ultra high molecular weight polyethylene or PEEK to provide minimal flexibility and corresponding motion of the components of the vertebral rod system, or can be fabricated from polyurethane or silicone to provide substantial flexibility and corresponding motion of the components of the vertebral rod system.

An elongated second section, such as, for example, a bone penetrating member 40 defines a longitudinal axis a and is configured for fixation with vertebrae V. Member 40 includes a circumferential wall 42 that defines an interior cavity 44 configured for disposal of flexible member 30 such that distal end 20 is disposed within interior cavity 44. Outer surface 32 has a continuous and non-interrupted configuration and is disposed in close fitting engagement with wall 42. It is contemplated that outer surface 32 may be non-continuous and interrupted, such as, for example, slotted, perforated, dimpled and/or undulating.

Wall 42 is circumferentially disposed about the entire distal end 20. Member 40 has an outer surface 46 that is threaded for fixation with bone. It is contemplated that member 40 may include alternate bone fixation elements, such as, for example, a nail configuration, barbs, and/or expanding elements.

Outer surface 32 engages wall 42 in a configuration such that shank 14 is pivotable to a plurality of axial orientations relative to axis a. In this configuration of member 40 and shank 14 with flexible member 30 being disposed therebetween in close fitting engagement, flexible member 30 also facilitates axial translation of shank 14 relative to member 40, and provides damping to bone fastener 10 and other components of the vertebral rod system upon application of stress, including flexion, extension and/or torsion to the vertebral rod system.

It is contemplated that member 40 can be variously dimensioned, for example, with regard to length, width, diameter and thickness. It is further contemplated that the respective cross-sectional geometry of member 40 may have various configurations, for example, round, oval, rectangular, irregular, consistent, variable, uniform and non-uniform. Member 40 may have a different cross-sectional area, geometry, material or material property such as strength, modulus or flexibility relative to shank 14.

In a first orientation of bone fastener 10, member 30 is disposed between shank 14 and member 40 such that shank 14 is longitudinally aligned with member 40 along longitudinal axis a. It is contemplated that in the first orientation, no flexion, extension or torsional forces are applied to bone fastener 10 and/or vertebral rod 12. As the components of bone fastener 10 move to a second orientation from the first orientation, flexion, extension and/or torsional forces are applied to bone fastener 10 such that shank 14 is pivotable to a plurality of axial orientations and moveable in axial translation, relative to axis a of member 40. It is envisioned that such relative pivotable movement of shank 14 includes bending through angle a relative to axis a, rotation in the direction of arrows x and axial translation in the direction of arrows y.

Flexible member 30 extends outwardly from cavity 35 to facilitate movement of shank 14. It is contemplated that member 30 may extend along a greater length of shank 14 or recess within cavity 35. It is further contemplated that distal end 20 may be disposed to a depth within cavity 35 to engage closed end 38, to a mid-portion of cavity 35 or only adjacent to a proximal portion 48 of member 40. It is envisioned that the greater depth that a tip 50 of distal end 20 is disposed within cavity 35, the less flexibility and range of movement shank 14 has relative to member 40, and alternatively, when tip 50 of distal end 20 is more closely disposed adjacent proximal portion 48, the greater flexibility and range of movement shank 14 has relative to member 40. Movement of the components of the vertebral rod system between one or a plurality of orientations is contemplated and may include a range of increasing and decreasing levels of resistance of the components of the vertebral rod system.

In assembly, operation and use, the vertebral rod system including bone fastener 10 is employed with a surgical procedure for treatment of a spinal disorder affecting a section of a spine of a patient, as discussed herein. The bone fastener 10 may also be employed with other surgical procedures. Bone fastener 10 is employed with a surgical procedure for treatment of a condition or injury of an affected section of the spine including vertebrae V, as shown in FIGS. 3-4. It is contemplated that the vertebral rod system including bone fastener 10 is attached to vertebrae V for fusion and/or dynamic stabilization applications of the affected section of the spine to facilitate healing and therapeutic treatment, while providing flexion, extension and/or torsion capability.

In use, to treat the affected section of the spine, a medical practitioner obtains access to a surgical site including vertebra V in any appropriate manner, such as through incision and retraction of tissues. It is envisioned that the vertebral rod system including bone fastener 10 may be used in any existing surgical method or technique including open surgery, mini-open surgery, minimally invasive surgery and percutaneous surgical implantation, whereby vertebrae V is accessed through a micro-incision, or sleeve that provides a protected passageway to the area. Once access to the surgical site is obtained, the particular surgical procedure is performed for treating the spinal disorder. The vertebral rod system including bone fastener 10 is then employed to augment the surgical treatment. The vertebral rod system including bone fastener 10 can be delivered or implanted as a pre-assembled device or can be assembled in situ. The vertebral rod system may be completely or partially revised, removed or replaced, for example, replacing rod 12 and/or one or all of the components of bone fastener 10.

Vertebral rod 12 has a rigid, arcuate configuration. A first bone fastener 10 is configured to attach an upper section 52 of vertebral rod 12 to vertebra V₁. A second bone fastener 10 is configured to attach a lower section 54 of vertebral rod 12 to adjacent vertebra V₂. Pilot holes are made in vertebrae V₁, V₂ for receiving first and second bone fasteners 10. Each penetrating member 40 of first and second bone fasteners 10 includes threaded bone engaging portion 46 that are inserted or otherwise connected to vertebrae V₁, V₂, according to the particular requirements of the surgical treatment. Each head 22 of first and second bone fasteners 10 includes through opening 24 configured to support rod 12, and a set screw 56, which is torqued onto sections 52, 54 to attach rod 12 in place with vertebrae V, as will be described. It is envisioned that vertebral rod 12 may have a semi-rigid or flexible configuration.

As shown in FIG. 4, the vertebral rod system includes two axially aligned and spaced rods 12, with sections 52, 54 extending through bores 24 of heads 22. Set screws 56 of each head 22 are torqued on the end portions of rods 12 to securely attach rods 12 with vertebrae V₁, V₂. Upon fixation of the vertebral rod system with vertebrae V, bone fasteners 10 each are configured to provide relative pivotable movement, rotation and axial translation of shank 14, as described above, as well as damping resistance to movement of the components of the vertebral rod system during flexion, extension and/or torsion of the spine.

For example, in an unloaded state of bone fasteners 10 and vertebral rods 12, which corresponds to the first orientation of bone fastener 10 discussed above, there are no appreciable tensile, compressive or torsion loads on vertebrae V₁, V₂. In flexion, extension and/or torsion of vertebrae V caused by corresponding movement of the patient, bone fasteners 10 react with relative movement of shank 14 and/or damping resistance that facilitates flexibility of rods 12 to a second, third or more orientation(s).

During movement of vertebrae V, for example, in flexion, extension and/or torsion, shank 14 moves relative to member 40 to facilitate relative flexibility and/or movement of rod 12 and/or other components of the vertebral rod system. Shank 14 pivotably rotates or bends through angle a relative to axis a, rotates in the direction of arrows x and/or axially translates in the direction of arrows y, relative to member 40, as shown in FIG. 3. This configuration increases resistance and provides damping during relative movement of shank 14 in a configuration that minimizes stress and facilitates shank 14 movement to avoid failure of bone fastener 10 including, for example, component fracture and/or loosening.

Bone fastener 10 may be employed as a bone screw, pedicle screw or multi-axial screw used in spinal surgery. It is contemplated that bone fastener 10 may be coated with an osteoconductive material such as hydroxyapatite and/or osteoinductive agent such as a bone morphogenic protein for enhanced bony fixation. Bone fastener 10 can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at the end portions of rod 12.

It is envisioned that the vertebral rod system described above including bone fastener 10 may be employed with a vertebral rod having an arcuate configuration and an increased length providing the ability to extend over two or more intervertebral elements. It is contemplated that the configuration of the vertebral rod system may provide load sharing, dynamic and/or flexible stabilization over a plurality of intervertebral levels, including treated and untreated vertebral and intervertebral levels.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A bone fastener comprising:
an elongated first section having a distal end;
a flexible member having an outer surface and defining an inner cavity configured for disposal of the distal end of the first section; and
an elongated second section defining a longitudinal axis and including a wall that defines an interior cavity configured for disposal of the flexible member such that the distal end of the first section is disposed within the interior cavity of the second section, the outer surface of the flexible member engaging the wall in a configuration such that the first section is pivotable to a plurality of axial orientations relative to the longitudinal axis of the second section.

2. A bone fastener according to claim 1, wherein the first section has a proximal end defining a cavity configured for disposal of a bone construct.

3. A bone fastener according to claim 1, wherein the distal end of the first section includes barbs.

4. A bone fastener according to claim 1, wherein the flexible member has a continuous, non-interrupted, surface that defines the inner cavity.

5. A bone fastener according to claim 1, wherein the flexible member has a continuous, non-interrupted inner surface that defines the inner cavity, the inner surface of the flexible member being disposed in close fitting engagement with an outer surface of the distal end of the first section.

6. A bone fastener according to claim 1, wherein an inner surface of the flexible member is circumferentially disposed about an entire outer surface of the distal end of the first section.

7. A bone fastener according to claim 1, wherein the outer surface of the flexible member is continuous and non-interrupted and disposed in close fitting engagement with the wall of the second section.

8. A bone fastener according to claim 1, wherein the wall of the second section is circumferentially disposed about the entire distal end of the first section.

9. A bone fastener according to claim 1, wherein the first section is movable in axial translation relative to the second section.

10. A bone fastener according to claim 1, wherein the flexible member is configured to provide damping.

11. A bone fastener comprising:
a shank having an outer surface, a proximal end and a distal end;
a flexible member having an outer surface and an inner surface defining an inner cavity having a closed distal end, the inner cavity being configured for disposal of the distal end of the shank and the inner surface engaging the outer surface of the shank adjacent the distal end; and
a penetrating member defining a longitudinal axis and including a circumferential wall that defines an interior cavity configured for disposal of the flexible member such that the distal end of the shank is disposed within the interior cavity of the penetrating member, the outer surface of the flexible member engaging the wall such that the flexible member is configured to provide damping.

12. A bone fastener according to claim 11, wherein the flexible member engages the wall in a configuration such that the shank is pivotable to a plurality of axial orientations relative to the longitudinal axis of the penetrating member.

13. A bone fastener according to claim 11, wherein the shank is movable in axial translation relative to the penetrating member.

14. A bone fastener according to claim 11, wherein the proximal end of the shank defines a cavity configured for disposal of a bone construct.

15. A bone fastener according to claim 11, wherein the distal end of the shank includes barbs.

16. A bone fastener according to claim 11, wherein the inner surface of the flexible member has a continuous, non-interrupted configuration that defines the inner cavity of the flexible member.

17. A bone fastener according to claim 11, wherein the wall of the penetrating member is circumferentially disposed about the entire distal end of the shank.

18. A vertebral construct comprising:
at least two bone fasteners, each bone fastener comprising:
a shaft having a distal end and a proximal end defining a cavity;
a flexible member having an outer surface and defining an inner cavity configured for disposal of the distal end of the shaft, and
a penetrating member defining a longitudinal axis and including a wall that defines an interior cavity configured for disposal of the flexible member such that the distal end of the shaft is disposed within the interior cavity of the penetrating member, the outer surface of the flexible member engaging the wall in a configuration such that the shaft is pivotable to a plurality of axial orientations relative to the longitudinal axis of the penetrating member; and
at least one vertebral rod having a first end and a second end, the first end being supported with the cavity of the proximal end of a first bone fastener and the second end being supported with the cavity of the proximal end of a second bone fastener.

19. A vertebral construct according to claim 18, wherein the shaft is movable in axial translation relative to the penetrating member.

20. A vertebral construct according to claim 18, wherein the flexible member is configured to provide damping.
